(19) United States
(12) Patent Application Publication
(10) Pub. No.: US 2004/0082980 A1
(43) Pub. Date: Apr. 29, 2004

(54) Programmable neurostimulator

(76) Inventors: Jaouhar Mouine, Sherbrooke (CA); Zied Chtourou, Montreal (CA)

Correspondence Address: David D Bahler, Fulbright & Jaworski, Suite 2400, 600 Congress Avenue, Austin, TX, 78701, US

(21) Appl. No.: 10/399,609

(22) PCT Filed: Oct. 19, 2001
(86) PCT No.: PCT/CA01/01490

(30) Foreign Application Priority Data
Oct. 19, 2000 (CA) 2,323,983

(51) Int. Cl. A61N 1/18

(52) U.S. Cl. 607/48

(57) ABSTRACT

A programmable neurostimulator and methods, characterized by its modular design is provided, comprising: an internal part based on a mixed-signal ASIC giving access to a complete control on the injected charges, and new channel concept allows performing any stimulation strategy and permit to use any stimulation mode (monopolar, bipolar, quadripolar . . . ); and an external part, built around a DSP and having a digital architecture, which allows to program any signal-processing algorithm, and to store different algorithms and stimulation strategies to be used and selected by the patient himself. An algorithm enables the use of as many filters as needed and the selection of their characteristics and of their associated channels. A vector quantization based stimulation method uses a finite set of element sounds defining all speech characteristics, in such a way that the patient is exposed to a limited number of stimulation sequences allowing the complete identification of the speech phonemes. A wavelet packet based stimulation method is developed towards a new multi-rhythm multi-resolution stimulation strategies that have never been used before. An appropriate hardware as well as a very user-friendly interface clinical s ftware supports all these aspects.

### FIELD OF THE INVENTION

The present invention relates to neurostimulators. More specifically, the present invention is concerned with a programmable and highly versatile neurostimulator such as, for example, a cochlear prosthesis system, and with improved stimulation algorithms including multi-rate and multi-resolution stimulation strategies.

### BACKGROUND OF THE INVENTION

Hearing disorders are generally classified into two categories: conductive hearing loss and sensorineural hearing loss. The former is associated with the conductive structures of the ear, namely the eardrum and the bones of the middle ear. Therefore, it originates in the outer and middle ears. Since these structures of the ear deal specifically with the amplification of sound, conductive hearing defects are generally remedied by conventional amplifying hearing aids. The latter, sensorineural hearing loss, is the result of a malfunction of hair cells within the cochlea (inner ear), fibers of the auditory nerve, superior nuclei and relays, or the auditory cortex of the brain. Sensorineural hearing loss can result from illness (for example, scarlet fever or meningitis), presbycusis, exposure to very loud noise (a blast or an explosion), working in noisy environments, ototoxic drugs, or genetic predisposition.

Approximately 10% of the world population suffer from hearing loss. Among them, about 10% are profoundly or totally deaf. Besides conventional hearing aids, these people can be helped by cochlear prosthesis.

Generally stated, cochlear prosthesis convert sounds into electrical pulses that are delivered to the endings of the auditory nerve in the cochlea, a function normally carried out by the hair cells to which these nervous fibers are connected within the inner ear. Obviously, this kind of device is efficient for people still having residual auditory nerve fibers together with a healthy upper nervous system. Hopefully, these represent the majority of cases. Unfortunately, more impaired people have much less options to overcome their hearing problems.

As illustrated in FIG. 1, the basic components of cochlear prosthesis are:

an external part 20 including a sound analyzer 22, a microphone (not shown) externally worn by the patient; a coding and modulation module 24;

an internal part 26 including a stimulus generator 28, surgically implanted under the skin behind the ear; a demodulation and decoding module 30; and an electrode array 32 that delivers electrical pulses to the auditory nerve fibers; and

a communication link 34 between the external and the internal parts.

Over the last two decades, a number of cochlear prosthesis have been developed to help profoundly deaf people overcome their hearing loss. Such systems incorporate either a single electrode or a multielectrode array, these electrodes being extracochlear, intracochlear, or modiolar. Considering the large number of hair cells, which generate the nervous influx on the auditory nerve and their organization, it is obviously difficult to assign an electrode to each hair cell. Additionally, because of technological (electrode fabrication) and safety (current density) considerations, the number of electrodes must be limited. However, the exact number of required electrodes is still unprecisely known. Different systems use different number of electrodes, thus providing different ability to control the direction and various distribution patterns of electrical charges, according to their electrode fabrication technique, and/or to their stimulation algorithm.

It is now well established that multichannel devices offer much better performances in speech comprehension than do single channel ones. Moreover, intracochlear electrode arrays are now commonly used unless there is anatomic counter indication such as cochlear ossification, in which case extracochlear electrode arrays are preferred. The ease of installation and the location of electrodes close to the nerve endings justify such a choice, which allows the use of lower stimulus levels and therefore power savings. As far as the communication link 34 is concerned, transdermal inductive links are preferred to percutaneous plugs for obvious safety and aesthetic reasons.

Furthermore, some of these systems generate monopolar stimulation while others rely on bipolar stimulation. The first stimulation mode consists in using a reference electrode, located relatively far from the active electrode or from the stimulation site, so as to allow spreading electrical charges over a large area, therefore affecting a large number of nerve fibers. This is usually needed when the number of residual auditory nerve fibers is limited. The second stimulation mode is characterized by the use of two electrodes located close to each other, and so configured that one of them is a source while the other acts a sink. This is usually used to generate electrical activity over a localized area, thus affecting a specific sample of nervous fibers.

While current cochlear prostheses are composed of the above-mentioned basic constituents, they differ in the number of electrodes used, in the stimulation algorithms adopted, and in some ergonomic features. These differences in turn result in different hardware designs for performing desired operations.

It is believed that the success of cochlear prosthesis primarily depends on the stimulation algorithm. Beside being executable by its hosting hardware, a stimulation algorithm is required to meet two basic criteria in order to be a viable stimulation algorithm: firstly, the processing time should be short enough to permit a real-time execution, and, secondly, the level of complexity should be reasonable, so that the stimulation algorithm can be implemented on a portable sound analyzer.

Ever since the first experiments were performed in the field, the stimulation algorithms have been based on two basic approaches. The first approach consists in extracting the speech features that are considered to be essential for the comprehension of speech (pitch, one or two formants), and then in formatting them according to the basilar membrane tonotopy. This approach places its emphasis on the frequency of the signal. The second approach is a wide-band processing of the speech signal. It consists in transforming the speech signal into different signals that are transmitted directly to the concerned regions of the basilar membrane. This approach places its emphasis on the temporal details of the speech signal.

Each one of the above-mentioned two approaches provides some level of speech perception. The technique of features extraction has demonstrated better performances in vowel identification, while the wide-band technique has given better results in consonants and open-set speech discrimination. Additionally, many specialists agree that the technique of speech features extraction affects the natural aspect of the acoustic signal and is sensitive to the surrounding noise. Moreover, it has been proven that the results of speech features extraction are very sensitive to small shifts in the localization of stimulation site on the basilar membrane, and not noticeably enhanced with increased stimulation rate. On the contrary, in the case of the wide-band technique, a direct correlation is demonstrated between the stimulation rate increase and the discrimination performances.

Currently available cochlear prostheses have been designed according to specific stimulation algorithms based on one of the two approaches mentioned hereinabove. Consequently, while providing some level of speech perception to many profoundly deaf people, they are definitively far from achieving the ultimate goal of complete speech comprehension. Moreover, the design of current prostheses is so closely dependent on the stimulation algorithm used that, generally, they can not be used to perform various new tasks without major hardware changes or surgical replacement of the implanted part. It is believed that this feature is a major obstacle to their evolution. The underlying reality is that current devices are unable to correctly emulate the auditory system functions.

In fact, despite a number of technological improvements, current prostheses fundamentally rely on the same basic twenty-year old principles of operation, and their stimulation algorithms have only slightly evolved. Furthermore, these technological improvements were mainly focused on size and power consumption reduction, by using advanced integrated circuit technology. This has even allowed the design of new “behind the ear” speech processors that are indeed much reduced in size, but also more devoid of versatility.

Not knowing the exact way the information is coded over the auditory nervous system, and in the absence of tools and experiments permitting to investigate further such matter, the manufacturers keep enhancing the features of their systems, without modifying the stimulation approach. For instance, on the one hand, the speech features extraction system manufacturers emphasize the importance of frequency resolution in the speech comprehension, claiming it enables low stimulation rates (and thus power savings) and reduced channel interaction, thus giving more possible stimulation channels. Their research aims at improving their system immunity to noise. On the other hand, when offering systems based on wide-band processing, manufacturers emphasize the importance of time resolution. Their research is then targeted to provide higher stimulation rates.

Regardless of the stimulation approach used, a shared concern is to simplify the surgical insertion of the electrode array, by providing new products based on advanced fabrication techniques, and to reduce the size of the devices so that they can be easily implanted.

It is apparent that, from a marketing point of view, these developments aim at providing more people with prosthesis, be they seriously hearing impaired people, prelingually deafened people, perilingually deafened people or even very young children.

Traditionally, the target population for cochlear prosthesis comprised only postlingually deafened adults. Recently, it has been demonstrated that the performances of the implanted devices depends primarily on the duration of the deafness, i. e. possibly on the length of time the nerve was deprived of stimulation due to the auditory mechanism defect. Hence, the shorter the period of deafness, the less auditory deprivation there is, and the greater the benefit from artificial stimulation can be expected to be.

Additionally, experiments on young children, whose brain plasticity allows them to adapt easily to the system limitations in order to reproduce artificial nervous stimulation, have demonstrated that acceptable results are possible, though at the cost of long and hard rehabilitation periods.

As for prelingually and perilingually deafened people, cochlear prostheses remain their major asset despite the limited performances that may be enhanced with other means, such as lip-reading.

It is believed there are possible significant improvements to be made. In particular, all aspects of a sound signal should be considered, independently of its nature (to be independent of the mother tongue of the patient), in order to design systems more efficient in emulating the natural auditory system, or, at least, able to feed the nervous system with the maximum amount of information it can process.

Such improvements would certainly boost the use of cochlear prosthesis for all categories of deaf people, enlarge the number of potential candidates, minimize the rehabilitation period, and provide better support to ever earlier diagnosed patients. Hence, these people would get the opportunity of a quick social integration, and would enjoy a better quality of life, by reducing their dependence on others, and participating as equal members of society.

### OBJECTS OF THE INVENTION

An object of the present invention is therefore to provide an improved programmable neurostimulator.

### SUMMARY OF THE INVENTION

More specifically, in accordance with the present invention, there is provided a programmable neurostimulator comprising an external part and an internal part designed to be implanted in a patient's body; said external and internal parts being linked; said internal part having a plurality of outputs each connectable to an electrode, said outputs being independently configurable to create different channels; said channels being independently selectable and addressable.

According to another aspect of the present invention, there is provided a programmable neurostimulator comprising an internal part and an external part, wherein said internal part comprises a digital part and an analogue part, said digital part controlling said analogue part by executing a set of command words and including a stimulation generator capable of generating a variety of stimulation waveforms by means of a variety of stimulation algorithms selectable by a patient, and wherein said external part comprises a sound analyser capable of extracting a variety of sound aspects.

According to a third aspect of the present invention, there is provided a stimulation method for a neurostimulator, wherein said stimulation method being based on an algorithm making use of a plurality of filters and on a selection of characteristics of each one of said plurality of filters and of channels associated to said filters; and wherein said stimulation method generates a stimulation frequency for each one of said channel, so as to achieve a compromise between frequency and time resolutions; and wherein said stimulation method is automatically adaptable to a variety of characteristics of a detected sound as well as to a patient's condition and pathology.

According to yet another aspect of the present invention, there is provided a sound analyser, wherein said sound analyser uses a variety of stimulation algorithms, and is provided with:

a graphical window dedicated to psycho-acoustic tests, which allows determining mapping parameters that are used by algorithms;

a graphical window associated to each one of said variety of stimulation algorithms, which permits to set up respective specific parameters of said variety of stimulation algorithms;

wherein said graphical windows can communicate between each other for exchanging common specified data or interdependent set-ups, and said sound analyzer provides a systematic phoneme identification process based on operations with a well-defined and limited number of frequency spectra including sound and noise, and adapts to a variety of mother tongues and regional linguistic particularities.

It should be pointed out here that a difference is made in the present disclosure and in the appended claims between a stimulation strategy and a stimulation algorithm. In the following, a stimulation strategy relates to how selected aspects of the sound signal are represented in the inner ear, regardless of how they are extracted, whereas a stimulation algorithm is composed of a speech-processing algorithm sustaining a stimulation strategy.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1, which is labeled “prior art”, is a simplified bloc diagram of a conventional neurostimulator in the form of cochlear prosthesis;

FIG. 2 is a simplified bloc diagram of the internal part of a neurostimulator according to an embodiment of the present invention;

FIG. 3 is a simplified bloc diagram of a mixed-signal integrated circuit of the internal part of FIG. 1;

FIG. 4 is a simplified bloc diagram of a sound analyzer according to an embodiment of the present invention;

FIG. 5 is a mapping graphical interface window used according to an embodiment of the present invention;

FIG. 6 is a VCIS graphical interface window used according to an embodiment of the present invention;

FIG. 7 is a binary tree representation of the wavelet packet decomposition used according to an embodiment of the present invention;

FIG. 8 is an illustration of the time-frequency compromise for multi-resolution analysis used according to an embodiment of the present invention; and

FIG. 9 is a wavelet packet based graphical interface window as available in an embodiment of the present invention.

### DESCRIPTION OF THE PREFERRED EMBODIMENT

All of the above-mentioned problems are dealt with in the present invention, together with the constraint of size, to provide a device that can be easily implanted without loosing any aspect of a complete versatility and a full programmability.

In short, the main features of the system of the present invention are its versatility in use, and complete external software programmability, making it completely “transparent” to any stimulation algorithm.

In accordance to a general aspect of the present invention, the approach used to achieve these features consists in considering each functional part independently of the others, and designing them to work in the most general way without any constraints imposed by the other parts. To ensure a complete versatility, each basic part is co-designed by a software algorithm running on an appropriate hardware platform.

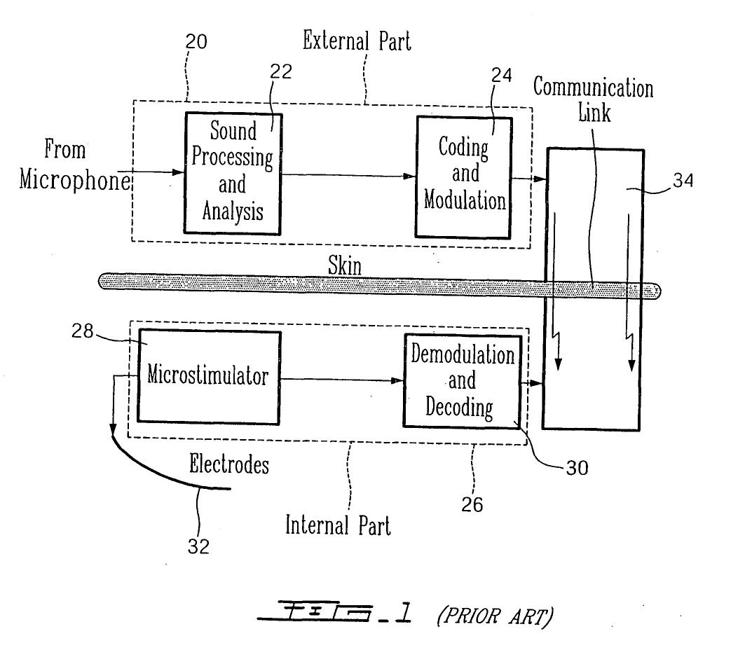

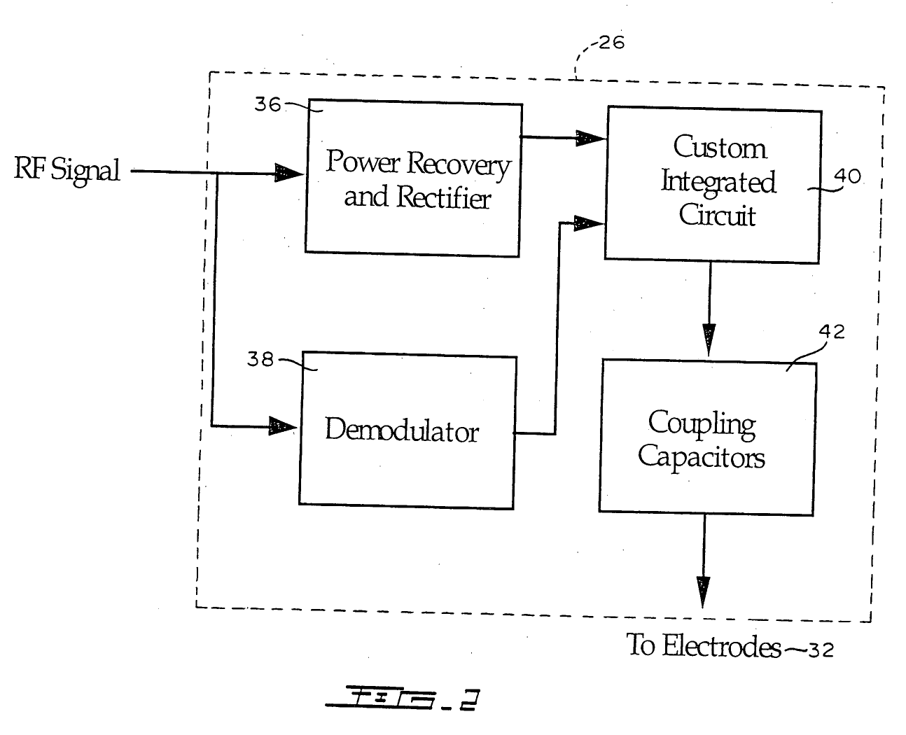

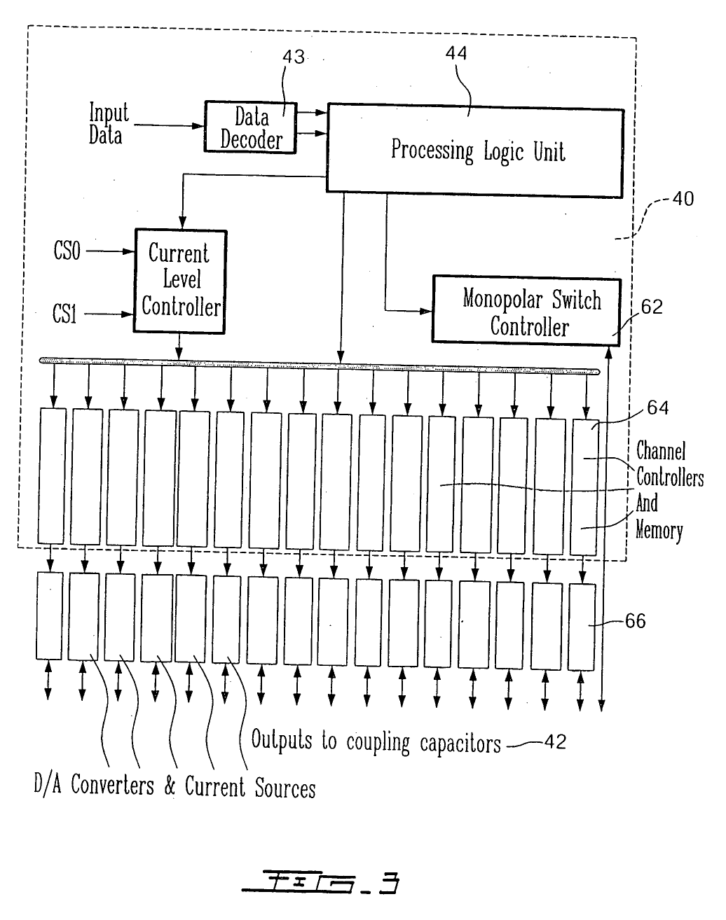

It is to be understood that the neurostimulator of the present invention, when in the form of a cochlear prosthesis, is generally as described hereinabove with reference to FIG. 1. Indeed, the neurostimulator of the present invention comprises an internal part and an external part. Turning to FIGS. 2 and 3 of the appended drawings, the internal part of the neurostimulator of the present invention will now be described.

In a preferred embodiment described herein, the internal part is built around a full custom application specific integrated circuit (ASIC) having a mixed-signal structure. The ASIC comprises a digital portion and an analog portion. The digital portion consists in a dedicated architecture executing a set of command words to control the analog part, which includes current sources, to generate stimuli and to perform desired operations.

More specifically, FIG. 2 shows a bloc diagram of the internal part 26. The internal part 26 appears as a hybrid circuit, basically made of a power recovery and rectifier module 36; a demodulator module 38; a custom mixed-signal integrated circuit 40; and a set of coupling capacitors 42, interconnecting the circuit 40 and the electrodes 32.

The power recovery and rectifier module 36 essentially comprises diodes and transistors used in rectifying the carrier wave coming from the link 34. On the other hand, the demodulator module 38 is used for demodulating the RF signal and extracting the incoming data.

The mixed-signal integrated circuit 40 receives serially transmitted data at say a 1M bits/second baud rate or higher. This rate permits to generate stimulation frequencies as high as 15625 Hz and more, and thus allows emphasizing on temporal details when needed, as in the case of stimulation algorithms based on wide-band processing of the sound signal. The output stimulus is a current waveform rather than a voltage waveform, which enables a better control of the injected charge quantity, since it is then independent of the biological tissue impedance.

Since it is a shared belief that the ear cannot distinguish more than 32 different stimulus levels, the circuit 40 is provided with 16 outputs, each giving access to 32 different current levels. The circuit 40 of the present invention delivers 32 different current levels over one of four current ranges that can be selected by the hardware.

The custom integrated circuit 40 will now be described in reference to FIG. 3, which is a simplified block diagram thereof. The integrated circuit 40 receives serially transmitted coded data containing command words. A decoder 43 extracts both data and synchronous clock. The data are then sent to a processing logic unit 44 to generate the appropriate control signals for the other parts of the circuit.

A current level controller 58 is used to provide an 8-bit accuracy, 32 current levels, ranging from zero to a maximum value depending on the setting of the two external signals (CSO and CS1). A monopolar switch controller 62 allows connecting the reference electrode to the ground when the monopolar stimulation mode is used. All of the processing operations are well synchronized and timed to perform real-time execution.

All of the control signals, the electrode address and the current levels are then sent to the channel controllers and memories 64 and to the D/A converters and current sources module 66 to perform the desired operation over the outputs. As shown on FIG. 3, each output has its own current level memory and its own controlling logic. The output signals of these control units are then applied directly on the transistors' gates of the eight-level digital to analog converter and the current source of each output. In that way up to 16 channels can be activated simultaneously. Moreover, to maximize versatility, the 16 outputs of the integrated circuit 40 may be selected and/or activated in any conceivable combination or manner, permitting to address any channel, set, or subset of channels independently of each other.

Let us explain here what a channel means in the context of the present invention. If a channel is associated to an electrode, every multielectrode implant is considered as a multichannel implant, regardless of the number of its current sources, and even if it has only one current source that can be switched over different electrodes. According to this first definition, the number of channels corresponds to the number of stimulation sites. In a second definition, a channel is associated to a charge distribution, which means that any current path generated between electrodes represents a stimulation channel. In the present invention, a channel refers to a current output provided with its own independent control unit, memory and current source.

In the present case, each channel can be addressed to generate its own given current level, or to be set in a specific mode, independently of the state or location of any other channel. Then, according to the second definition given hereinabove, more than 65 535 channels corresponding to different combinations of electrodes can be obtained, which result in different current paths or charge distributions, without any temporal or spatial constraints. Hence, each output can be configured as a current source, a current sink, a ground, or set in high impedance state independently of the others. In that way, it is possible to perform monopolar, bipolar, quadripolar, or any other stimulation mode. Of course, all these possibilities are accessible from external software programming, without any hardware limitation requiring replacing the internal part. Thus, this allows the generation of any stimulus waveform of any shape and any current distribution.

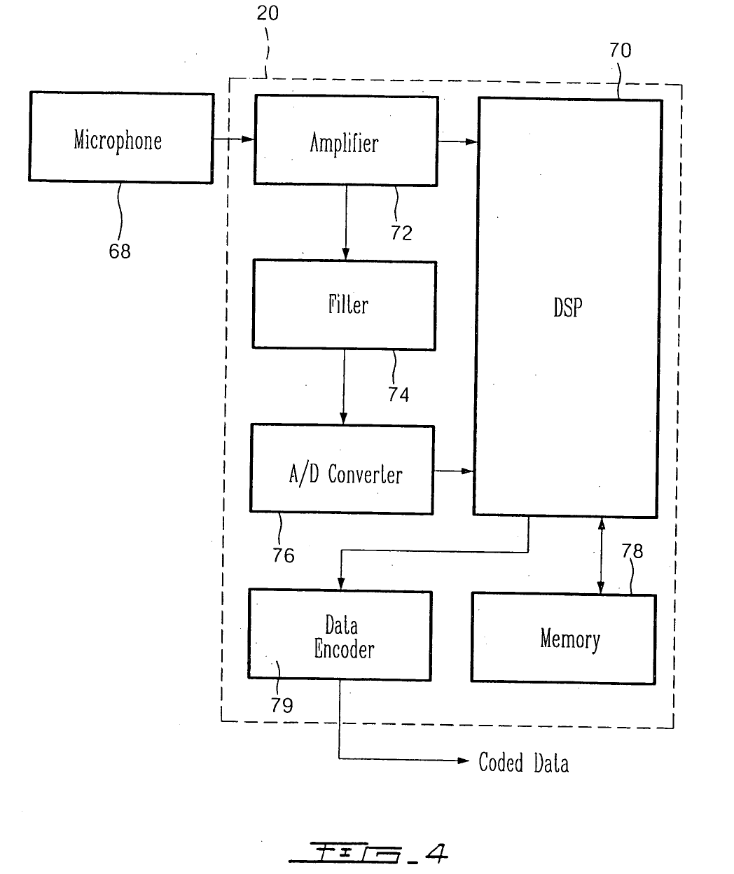

The external part of the system will now be described with reference to FIG. 4.

The external part 20 mainly comprises a microphone 68, an amplifier 72, a filter 74, an A/D converter 76, a Digital Signal Processor (DSP) 70 provided with an internal memory, an additional external memory 78, a data encoder 79 and various other small components (not shown).

The microphone 68 collects the sound signal, which is then amplified, filtered and converted in a digital signal, before being dispatched to the DSP 70.

Beside the internal memory of the DSP 70, an external flash memory 78 is added to store the boot software of the system, as well as all the parameters used in the stimulation algorithms, and the data needed to perform analysis of sound and electrical stimulation.

The external part 20 also comprises various other components (not shown) that stabilize and regulate the power for each module, an algorithm selector circuit that will be operated through an external switch, and some “glue logic” regrouped on a single Complex Programmable Logic Device (CPLD).

This CPLD is used to connect correctly the different parts of the system and to ensure functional operations. It allows the interfacing of the DSP with the flash memory 78, the A/D converter 76 and the external environment. This means that it contains a circuit to synchronize the serial transmission between the A/D converter 76 and the DSP, and to detect if another algorithm has been selected. It also contains the circuit performing the encoding of the output data to be dispatched to the internal part 20.

To ensure versatility, the external part 20 is designed in a modular way, by dividing its operation into four basic functional parts. Indeed, the overall system operation can be divided into different functional parts:

1. the operating system, which allows controlling general tasks;

2. the signal processing algorithm, used to analyze the sound and to determine its different aspects to be taken into account;

3. the stimulation strategy, used to represent any sound aspect in the inner ear; and

4. the encoding of the system output to be conveyed to the internal part. Anyone of these functional parts can be programmed independently of the others.

It should be pointed out here that a difference is made between a stimulation strategy and a stimulation algorithm. A stimulation strategy relates to how selected aspects of the sound signal are represented in the inner ear, regardless of how they are extracted, whereas a stimulation algorithm is composed of a speech-processing algorithm sustaining a stimulation strategy.

The external part 20 of the system can operate either in a stand-alone mode or in a slave mode. The first mode is usually used when the patient wears the system on a daily basis. The stand-alone mode assumes that the system has been well adjusted and programmed. On the contrary, in the slave mode, the system is linked to a computer, for example an IBM compatible PC (not shown), for performing tests, reprogramming, doing clinical experiments or setting up or adjusting the system.

The idea behind performing clinical trials by means of the portable system is to ensure that the desired operations are executable by the system in a stand-alone mode, and simultaneously, to provide the patient with a ready-for-use device as soon as the clinical tests are finished. It is to be understood that an appropriate PC interface (not shown) has been designed to communicate with the system.

The correct operation of the system is monitored by the DSP, which is the core of the system. The DSP can be boot loaded in two ways, since the speech processor's software can be downloaded either by using a serial boot or by using a parallel boot. The serial boot load is used to initialize a blank system and then is used when the system is connected to the PC. This allows programming the flash memory or setting the contents of some DSP's registers. The parallel boot can be performed directly from the on-board flash memory. This allows the download of the main operating system and the selected stimulation algorithm according to the algorithm selected by the patient.

The system is then ready to be used in a stand-alone mode. If another algorithm is selected, the DSP operation is interrupted to download the new selected algorithm from the flash memory and then the system resumes its normal operation. When a command is detected from the DSP serial port, this means that the system is connected to the PC and then it falls into a slave mode permitting to perform operations directly from the host computer. This normally happens at the time of performing clinical experiments, which would be followed by programming the flash memory to store new data issued from that test session.

Although the system hardware described hereinabove and developed for a greater versatility seems to be complex, the complete sound analyzer according to the present invention, including the required 4 AA rechargeable powering batteries, fits in a 90×60×25 mm package. This size is comparable to, and in some cases even smaller than, that of other available systems. Moreover, by incorporating the new integrated circuit technology, the system can be considerably reduced in size and will fit in a “behind the ear” package. The patient is allowed to enjoy the adaptability of the system hardware, without having to deal with its complexity. The only controls that he has to manipulate are the volume button and the algorithm selection switch as for any other system. On the other hand, due to the modular way it has been designed with, to its flexibility and versatility, as well as to its complete external programmability, it is believed that the system can be assembled in a completely implanted version.

We will now expand on a clinical software tool provided in the system of the present invention, as is usual in any conventional cochlear prosthesis.

A clinical software tool allows adjusting and programming the system according to the individual's pathology and physiological state. The clinical session is usually composed of two basic parts. The first part, known as the “mapping”, consists of a psycho-acoustic test that allows determining the effective functional stimulation channels, which will be or may be used, together with their corresponding dynamic ranges limited by the detection and pain (discomfort) thresholds. The second part consists of tests that allow adjusting the stimulation algorithm parameters according to mapping results.

For maximum versatility, the present clinical software has been developed on Microsoft's Windows™ platform, using object-oriented programming. This approach leaves the way open to future enhancements and upgrades, and is more appropriate for a modular structure that is meant to allow including future developments in the field, and providing versions that can be adapted to specific needs. The software consists of a very user friendly and completely graphical interface, which permits to give access to all stimulation parameters that may affect the perception of sound in the inner ear, taking advantage of the adaptability of the other parts of the system.

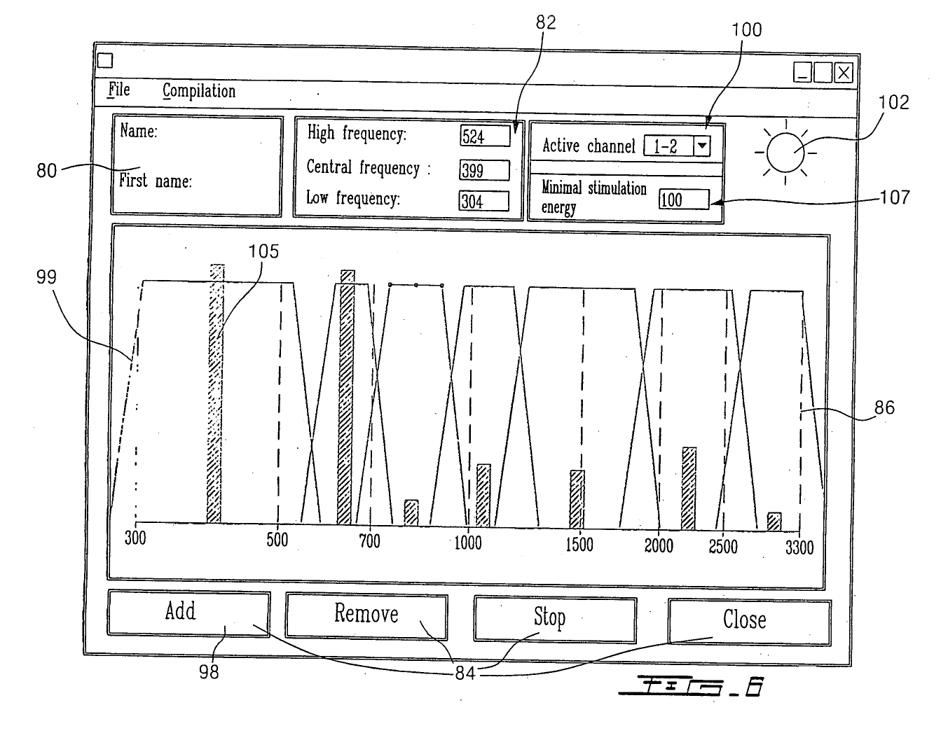

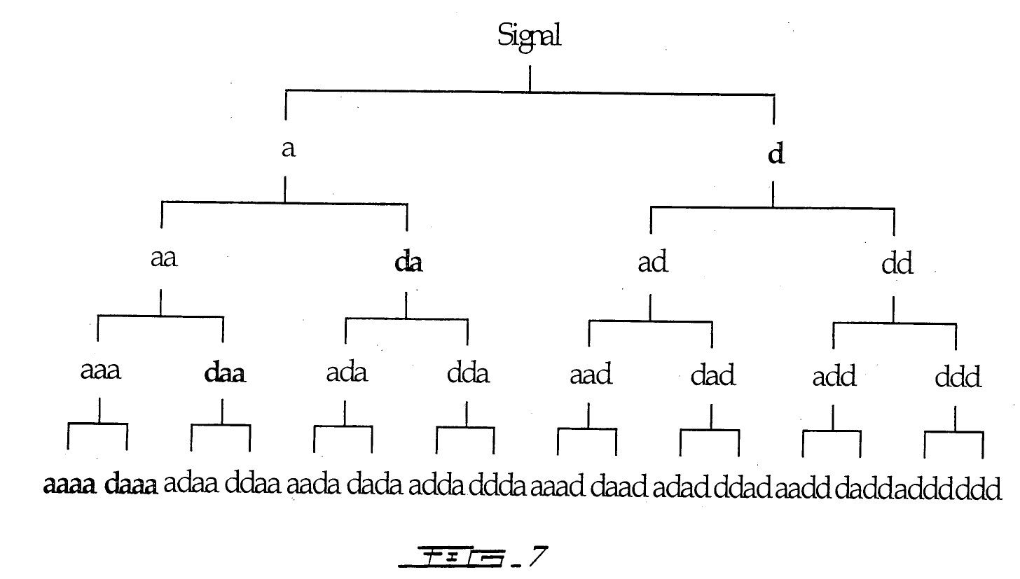

The modular structure is achieved by using different graphical windows, each one being associated to specific setups (FIGS. 6, 7 and 10). For instance, a window is dedicated to psycho-acoustic tests, which allows determining mapping parameters that are used by all of the stimulation algorithms. Similarly, a specific window is used for every stimulation algorithm, which permits to set up their respective specific parameters. The windows can communicate between each other for exchanging common specified data or interdependent set-ups. In such a fashion, the software can be adapted for use of only a single given algorithm by enabling only two windows (mapping and stimulation algorithm, see FIGS. 6 and 7). The software can then be extended anytime for implementing a new stimulation algorithm, by creating a new window that allows adjusting its parameters and setting its related specifications. Thus, it is possible to select a limited version to be used by patients at home for self-rehabilitation by disabling the psycho-acoustic test window, which ensures safety and prevents unintentional changes of the basic set-ups.

Currently, all cochlear prosthesis systems comprise a clinical software psycho-acoustic test part for adjusting the device to the patient physiological state according to the results of the surgical installation, i.e. depending on the final state and positioning conditions of the electrode array. Generally, since the available cochlear prostheses are designed in relation to a specific stimulation algorithm, the clinical software psycho-acoustic test part is also designed specifically in accordance to a given device. On the contrary, in the present invention, because the system is endowed with various capabilities, this part is designed independently of the number and of the address of the channels, and therefore can be used for any other available system.

The clinical software psycho-acoustic test part is intended to perform two basic operations. Firstly, it must define each functional stimulation channel that can, or should, be used. Then it should determine the dynamic range corresponding to each such stimulation channel, by setting, on the one hand, the minimum current level at which the patient starts to perceive sounds (referred to as the detection threshold), and, on the other hand, the maximum current level that can be supported by the patient without feeling any pain (referred to as the pain or discomfort threshold). Basically, this discomfort threshold depends on the number and on the condition of the patient's residual auditory nerve endings, and on the degree of insertion of the electrode array, which determines the localization of the stimulation sites with respect to the frequency partition of the basilar membrane.

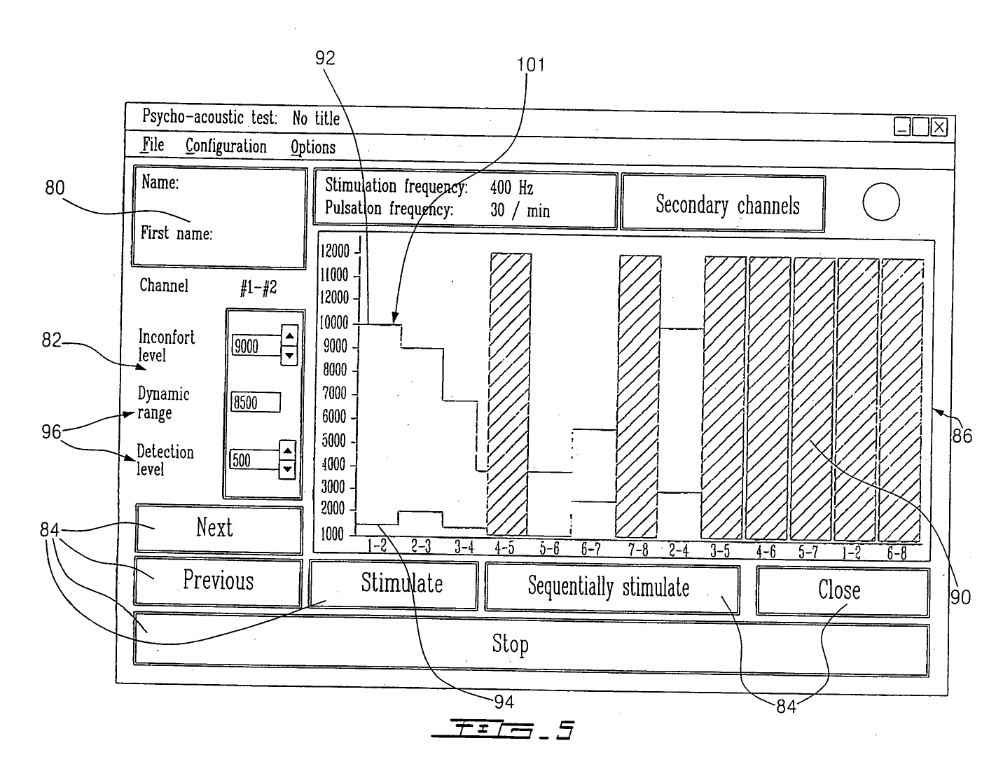

The window designed to perform this clinical step is shown in the appended FIG. 5. It contains a patient identification field 80, a display field 82 of the selected stimulation channel and parameters in use, a plurality of push buttons 84 to execute operations by simply clicking on with the mouse pointer, and a graphical representation 86 of stimulation channels.

To begin with, there are no predetermined stimulation channels. The user selects any electrode combination to set these channels in any desired stimulation mode (monopolar, bipolar, quadripolar, n-polar). For example, the most commonly used electrode combination associates each two adjacent electrodes to a bipolar stimulation channel. This set can then be identified as the set of primary stimulation channels, while a set of secondary stimulation channels is defined by associating each one to a pair of electrodes separated by one electrode, a set of tertiary stimulation channels is defined by associating each one to a pair of electrodes separated by two electrodes, and so on. A stimulation channel in use can be displayed on the screen and represented by a column 101 using a vertical scale to designate the current level that will be injected on. A channel that can not be used for any reason (for example, the missing of corresponding residual nerve fibers, or an electrode array defect) or that is intended to be disabled is also displayed on the screen and represented by a hatched column 90.

Once the different stimulation channels are defined, the physician proceeds to tests aiming at determining the data relative to each channel. A stimulation channel is enabled or disabled by turning it respectively to an active state or an inactive state by a simple click on the mouse's right button. This will make a dialog box to appear, where one can specify the state and the stimulation frequency to be used.

A distinctive aspect of the system of the present invention is that the stimulation frequency may be set to any value and can be varied from one channel to another. An active channel is selected for use by a click on the screen with the mouse. The column corresponding to such a selected channel comprises two horizontal stripes of different colors. The upper stripe 92 marks the corresponding pain (discomfort) threshold, while the lower one 94 refers to the corresponding detection threshold relatively to the vertical current level scale. These two thresholds encompass the dynamic range to be determined for each stimulation channel and to be used by the stimulation algorithms. The numerical value of the current level of each threshold is displayed at the left of the window 96. These values can be changed either by using arrows to increase or decrease them or by entering a new value in the corresponding field. To ensure the patient safety when the difference between the new value entered and the old one exceeds a maximum step value set by the physician, a warning box appears asking for confirmation of the operation. Once the dynamic ranges of all channels are determined, a sequential stimulation can be performed over all of them for comparing the different thresholds.

All of the data resulting from a psycho-acoustic test session are labeled with the patient's name and the date, and stored in a database to be used for future evaluation of the rehabilitation progress or to be used by the different stimulation algorithms.

Turning now to another aspect of the present invention, we will now describe the stimulation algorithms developed for the present system.

As already mentioned, currently available cochlear prostheses conventionally use stimulation algorithms based either on speech features extraction or on wide-band speech processing. In both cases, the processing is usually performed by using band-pass filters to extract the targeted feature or to decompose the speech signal. The technological solutions used to achieve that can be different from a system to another or from a version to another for the same system. However, even by using the most recent and the most advanced technologies, the principles remain the same and are characterized by a lack of adaptability, caused, for example, by the use of a fixed number of filters.

The present invention involves different stimulation algorithms, including an enhanced version of the classical ones as well as more advanced and promising ones taking advantage of the computing power of recent advanced technologies.

As explained hereinabove, a stimulation algorithm is composed of a sound processing algorithm and a stimulation strategy. The following sections will describe different sound processing techniques, which can be used with one or several stimulation strategies leading to different stimulation algorithms that can be implemented on the system of the present invention.

First, the classical technique, based on a filter bank, will be considered. In such a case, the present invention enables unlimited adaptability and complete programmability, since the system is digital and built around a DSP. Therefore, any available algorithm of stimulation, based either on speech feature extraction or on wide-band speech processing, may be programmed.

For purpose of illustration, the description will be based on the well-known CIS (Continuous Interleaved Stimulation) algorithm. However, it should be kept in mind that the present technique covers much more possibilities than the CIS algorithm.

The algorithm will be thereafter referred to as Versatile CIS (VCIS). In the CIS algorithm, the frequency band of the speech signal is split into six sub-bands of fixed frequency. Each one of these sub-bands is associated to a stimulation channel, and then the corresponding signal modulates a train of non-overlapping biphasic pulses that are delivered to the inner ear.

In the VCIS, there are no limits on the number of frequency sub-bands, and eventually no fixed central frequencies. The physician can use as much different frequency sub-bands as it is necessary, and can vary their bandwidths and central frequencies as he judges pertinent to the patient pathology according to the test session results.

For a better understanding of the versatility of this algorithm, the user graphical interface designed on the clinical software tool to perform the adjustments and programming of the system will be described in reference to FIG. 6.

The window associated with this interface includes the patient's identification field 80, the numerical values 82 of the filter characteristics and its associated channel, some push buttons 84 to execute operations by simply clicking on them, and a schematic graphical representation 86 of the frequency response of the filters. To add a new frequency sub-band, the physician has only to click on the “Add a Band” push-button 98. An additional trapezoidal shape 99, representing a new filter, appears on the central graphical area. The physician can then slide it by using its upper side central point while dragging the mouse and can stretch its upper corners to set the low and high frequencies of the filter. The numerical values of the selected frequencies then appear in the corresponding boxes, at the top of the window 82.

Once the filter parameters are chosen, the physician designates the stimulation channel to be associated to this filter among those available in the list box labeled “active channel” 100. This list contains only the channels that have been identified as viable and calibrated in the mapping session.

It is worth noting at this point that a given channel can be associated to any sub-band and to more than one sub-band. This feature allows to transposing the frequency contents corresponding to a defective fibers region on another region, and can accommodate a reversed cochlea or other possible anomalies of the inner ear.

Additionally, the physician can set the minimum acoustic energy 107 that should be reached before the received signal is considered a useful sound. This feature allows minimizing the surrounding noise effect.

Once all set ups are performed, the physician can proceed to the testing of the stimulation algorithm on the patient. While the stimulation is in progress, the flag 102 located in the top right corner of the window flashes and the relative signal energy of each sub-band is illustrated by modulations of the height of red bars 105 appearing at the location of the central frequencies of the different sub-bands. These two visual references are very helpful to monitor the system operations to find the desired frequency distribution of the sub-bands.

Finally, at the end of the rehabilitation session, the physician can program the system by downloading the stimulation algorithm into the portable sound analyzer, and also store the resulting data labeled with the patient's name and the date in the data base to be retrieved when needed.

Turning now to stimulation methods and techniques, the vector quantization based technique will be first considered.

Used in accordance to the present disclosure, this technique benefits from the computational power and large additional memory of the system of the present invention, especially as far as the speech-processing algorithm is concerned.

Basically, this method consists in performing a fast spectral analysis of each speech segment, and to compare the obtained spectra to those of a codebook stored in the system memory, in order to determine the one that is the best match. This codebook contains a limited number of sound identification elements, which are determined according to speech phonemes (for example, there are between 31 and 36 phonemes in the French language).

The execution time of the operation is very short, which ensures real-time processing. Once the speech segment is identified and associated to an element of the codebook, a corresponding stimulation sequence is generated in the inner ear through appropriate commands sent to the implanted part of the neurostimulator. For a given codebook (that may contains 128, 256, 512 or more spectra), there exist as many different stimulation sequences as the number of elements contained therein. These sequences are also stored in the system memory and each one of them is represented by a set of the microstimulator commands describing the stimulation strategy.

There are many advantages to use this technique of vector quantization based stimulation in cochlear neurostimulators. One advantage stems from the fact that the number of sounds or phonemes that the patient has to identify is limited to the codebook contents. This feature greatly facilitates the rehabilitation process, and allows the patient to get used rapidly to the sound identification, thereby resulting in a shorter period of reeducation.

Another advantage stems from the smoothness of the transmitted information, since the spectra corresponding to each phoneme are issued from a statistical average obtained from the same words pronounced by different people.

It should be pointed out here that, in conventional systems, the patient tries to identify the sound, for which a stimulation sequence has been generated, by considering the additive noise such as surrounding noise. This can explain the limited performances of these systems, since the additive noise depends on the conditions in which the sound is detected. In that respect, an advantage of the system of the present invention, when using the vector quantization technique, lies in a more systematic phoneme identification process, which is based on operations with a well-defined and limited number of frequency spectra (including sound and noise), hence considerably enhancing the signal to noise ratio.

Furthermore, since the stimulation sequences corresponding to elements of the codebook are stored in the programmable memory of the system, it is easy to use different memory fields for different stimulation strategies, and then to switch between them depending on the patient's preferences and performances. Such stimulation strategies may obey well-known psychoacoustic models, or may be established through empirical tests, performed on the patient, and then developed according to his preferences.

Interestingly, the present technique permits to adapt the stimulation sequences to the mother tongue of the patient, and even to his regional linguistic particularities. This means that a stimulation algorithm developed by using a given language can be easily adapted to other languages by simply downloading an appropriate codebook.

Since the system of the present invention is completely digital and built around a powerful DSP, all of the available stimulation algorithms, either based on speech features extraction or based on wide-band speech processing, can be programmed on it. This versatility, combined with that of the inner implanted part, permits a better representation of the speech signal in the inner ear.

Another stimulation technique will now be considered, namely the wavelet packet based technique. The two techniques previously described, referred to as the classical technique (described with reference to VCIS), and the vector quantization technique, respectively, make use of one or the other of the two basic approaches explained hereinabove (frequency aspect, temporal aspect), and are closely dependant of the sound to be coded that is the speech signal.

In contrast, the wavelet packet based technique described hereinbelow is based on the auditory system modeling and on the representation of the information in the auditory nerve, rather than on the sound source modeling. Therefore, it can be applied regardless of the nature of the sound. It attaches equal importance to both frequency and temporal aspects of the sound. This means that it permits the rate-place encoding of tonotopic information contained in the signal (frequency aspect), as well as the time-place encoding of the fine temporal information (temporal aspect).

The stimulation algorithm that is obtained with this approach achieves a compromise between frequency and time resolutions (multi-resolution), and is automatically adapted to the characteristics of a detected sound, as well as to each patient's condition and pathology.

In particular, in case where the sound signal contains many temporal details, such as in the case of non-stationary segments of the sound (consonants) for instance, the processing algorithm orders a high stimulation rate for better temporal resolution. In an opposite case, such as in the case of stationary segments of the sound (vowels), the processing algorithm orders low stimulation rates and more stimulation sites to achieve a better frequency.

Hence, by combining the respective advantages of two classical approaches described hereinabove, the present approach achieves simultaneously a high consonant discrimination, comparable to that obtained by the wide-band speech signal processing approach, and a high vowel discrimination of the order of that permitted by the speech signal features extraction approach.

As can be assessed by one skilled in the art, the way the present approach combines the benefits of the above-described approaches is not obvious. It consists in using them in a well-organized order and a well-defined way. For example, the high stimulation rates are to be used only when necessary, to prevent excessive current dissipation in the cochlea and thus allow power savings. Similarly, in the case of low stimulation rates, a higher number of stimulation channels is to be used with appropriate synchronization of their firing time and precise site or spatial coordinates corresponding to different frequency bands distributed all over the basilar membrane.

This judicious use of high and low stimulation rates (multi-rate) resulting from a good compromise between frequency and temporal representations is essential in improving the system performances and the limited speech comprehension results obtained by other systems. More details on the signal processing algorithm and the different stimulation strategies that could be used with it will be given hereinbelow.

Indeed, multi-resolution representation of the sound signal energy is proposed for analyzing the sound signal. It is based on a principle close to that enabling to locate a town on the globe: it is first located on a first scale, within a continent, then at a finer scale within a country, then within a province and so on until a scale allowing obtaining the most specific details of this town.

To understand the signal processing technique proposed in the present invention, the wavelet theory will first be introduced as the theoretical basis of this algorithm.

The basic idea behind using a processing technique based on the theory of wavelets to analyze the signal is to obtain information on the exact localization of the signal irregularities, in both time and frequency.

In the theory of wavelets, the signal is decomposed on a basis of functions that are concentrated both in time and in frequency. These functions, called wavelets, have all the same shape. They differ only by their size and their temporal location. The basic waveform used to generate these functions is called the mother wavelet. A signal can then be represented by the superposition of such functions, or wavelets, translated and dilated. The weights of such functions used in this decomposition, said wavelets coefficients, define the wavelet transform, which is then a function of two variables: the time and the scale (or dilation). In such a fashion, a representation of the energy of the signal is obtained in the form of an energy density depending on the scale (or frequency) and the time.

The wavelet transform as described above provides a signal representation that is redundant. In practice, the present invention makes use of a discrete version of this general transform, which is based on orthogonal function basis, and which minimizes redundancy and is more appropriate to digital signal processing.

This discrete wavelet transform has been used previously to generate a signal-processing algorithm based on multi-resolution analysis. This algorithm consists of using different scales to represent the signal, so that the signal is replaced by a different approximation in each scale. The signal representation is thereby all the more precise that the scale is smaller. The analysis is then performed by determining the difference between two successive scales, which is called the detail.

To implement this multi-resolution analysis algorithm, the signal is processed through successive stages, each one involving the so-called wavelet functions and scale functions. These functions are represented respectively by a high-pass filter and a complementary low-pass one. The high-pass filter output gives the detail at a given scale, whereas the low-pass filter output gives the approximation of the signal at the same scale. This approximation becomes then the input of the next stage. The outputs of each stage are down sampled to keep the same number of samples as in the input signal. The number of stages may vary depending on the desired precision.

It has been shown that the multi-resolution analysis algorithm just described is a particular case of a transform called wavelet packet. This transform is a generalization of the time-frequency analysis made by the wavelet transform. It consists in applying the wavelet functions and scale functions to both the approximation and the detail of each scale or stage of processing.

The process of the wavelet packet decomposition can then be represented by a binary tree, as shown in FIG. 7, that contains all possible function bases that may be used in order to process the signal. The choice of the appropriate function basis is made according to cost considerations based on specific performance criteria.

By this processing technique, the signal is analyzed in a way very similar to the biological processing of sounds performed within the inner ear.

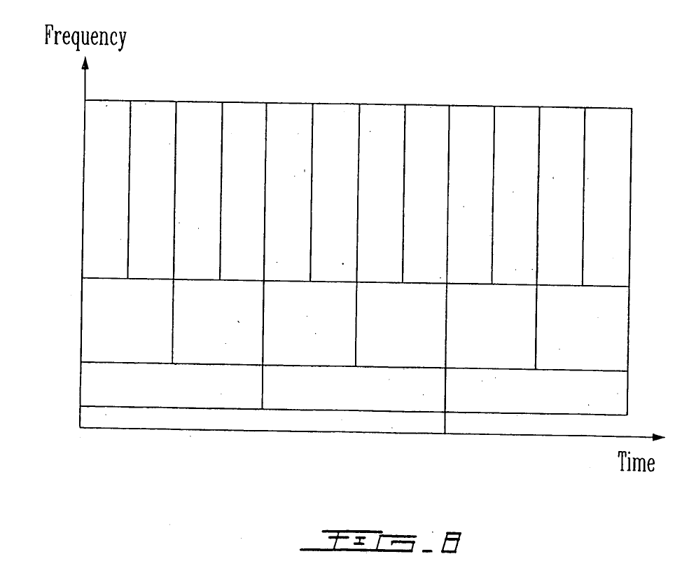

Thus, the high frequencies in the sound signal are analyzed through large frequency band windows, whereas low frequencies are analyzed through narrow frequency band windows. Correspondingly, in the time domain, this means that segments of sound that present a lot of variations are analyzed with a fine temporal scale, in order to characterize their rapid variations, while stationary sound signals are analyzed through coarse temporal scales (FIG. 8).

Differently stated, this method of processing analyses the sounds as being a succession of systems with an impulse response of a characteristic duration, which is inversely proportional to the scale used. This is closely related to the natural way that the information is decoded in the auditory nerve and can be described by different models that establish a relationship between the mechanical characteristics affecting each specific hair cell and the duration of this cell response.

Hence, the scale parameter, which fixes the duration of the decomposition function in the wavelet packet transform, is related to the site of the affected hair cell, which corresponds to the site of stimulation and to the position of the electrode within the cochlea. The second parameter defined by the time variable in the wavelet packet transform, called the delay parameter, automatically gives the exact time when the stimulation has to be sent on the different electrodes.

The energy density resulting from the wavelet packet decomposition depends on the choice of the mother wavelet. Ideally, this wavelet should have the same shape as the impulse response of a hair cell. In this way, the spanning of signals energy in the time-frequency plane will be similar to the spanning obtained if the cochlea is stimulated at the stimulation sites defined by the scale parameter, at the instants defined by the delay parameter, and with magnitude equal to that of the corresponding decomposition coefficient. Thus, it is possible to reproduce in the cochlea the normal wave glissando induced by the acoustic signal on the basilar membrane, as occurs in the natural process.

When dealing with artificial nervous stimulation used in cochlear implants, there is no way to determine the correspondence between stimulation site and frequency range, nor the impulse response of hair cells, which a priori may differ from a patient to another, depending on the encountered pathology and the electrode array insertion. Hence, there is no such thing as a general form for the mother wavelet, nor a fixed decomposition basis for the wavelet packet transform used. Consequently, while the clinical software of the present invention is supplied with several well-defined waveforms of mother wavelets, it can be provided with as many new mother wavelets as needed. At the time of performing tests on a given patient, the audiologist determines the more appropriate mother wavelet and the more efficient decomposition basis to be used. This depends on the patient's perception of sound, on the pathological state of his cochlea, and on the current state of the device surgical installation. Such an adjustment can take the form of a try and error process, supported by the comments from the patient.

The signal-processing algorithm proposed herein can be used with different stimulation strategies. Since the objective is to help recover hearing with a defective cochlea, complete freedom is left to the audiologist to represent the sound signal in different ways in the inner ear. In the following sections, we will describe different proposed stimulation strategies keeping in mind that there exist many others that can be programmed and used with the system of the present invention.

A first stimulation strategy will now be described, with reference to FIG. 7.

When progressing down the tree of FIG. 7 from a scale to the following, the number of stages is doubled, the frequency resolution gets higher and the number of samples, in each level of the decomposition, is kept the same as the number of original input samples. For instance, in the case of an acoustic signal with a frequency band of 4000 Hz and a length of N samples, there are two stages in the first level of the binary tree with 2000 Hz frequency band and N/2 samples each, four stages with 1000 Hz frequency band and N/4 samples each on the second level, and so on.

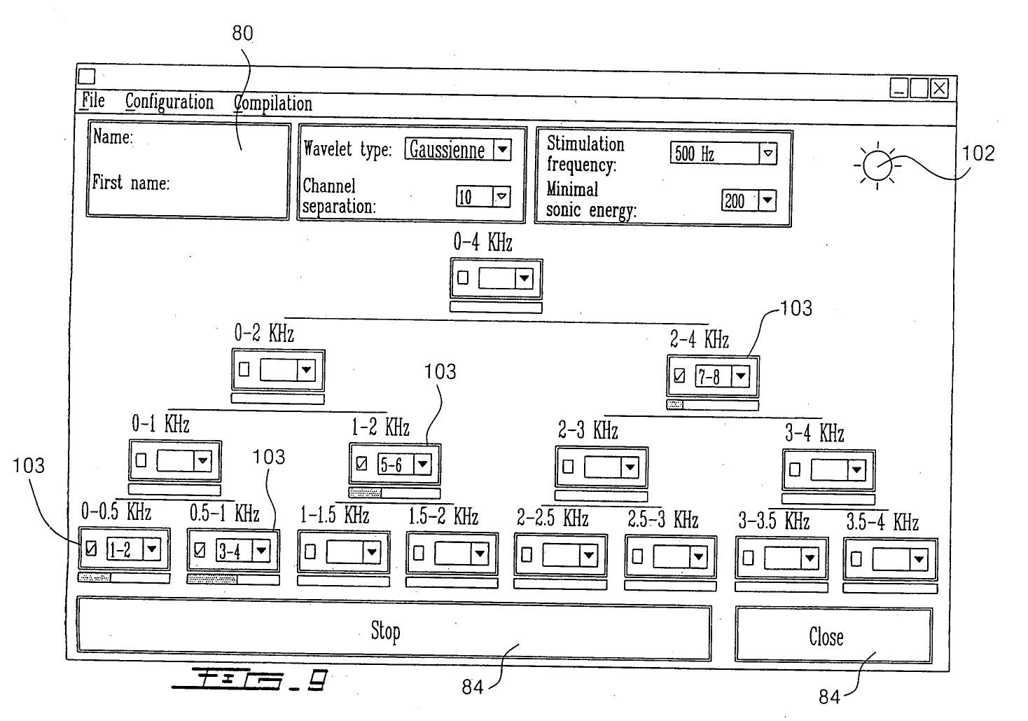

Each one of the stimulation channels 103 (shown in FIG. 9) is associated with a stage in the global decomposition tree. This association depends on the patient's perception and can be refined during different test sessions. This strategy uses different stimulation rates, from one level to the other. The rate of stimulation on each channel is fixed by the number of coefficients issued from the signal decomposition at the associated stage.

For example, if we consider a sampling frequency of 8 kHz, a channel associated with a stage in the first level of the decomposition tree will have stimulation rate of 4000 pulses per second. A channel associated with a stage in the second level will be stimulated at a rate of 2000 pulses per second. Finally, a channel associated with the third level will be stimulated at 1000 pulses per second rate.

Therefore, different temporal resolutions are possible for different stimulation sites or frequency ranges. The more the frequency content of a stage is important, the higher the stimulation rate is, and vice versa. The time and the order of stimulation on each channel are dictated by the coefficients of the wavelet packet decomposition at the associated stages, and on their temporal location.

A second stimulation strategy consists in a modified version of the first one, using a low common rate of stimulation. It is useful in cases where the patient does not bear the high stimulation rates of the first strategy. In this second stimulation strategy, only the maximal decomposition coefficient in each stage is used to modulate a pulse on the corresponding channel.

A third stimulation strategy makes a maximum use of the patient's dynamic range. In fact, the stimulus frequency affects perception. Hence, for each stimulation site on the cochlea it exists a certain stimulation frequency that offers the largest dynamic range. This frequency will be called, hereinafter, the channel's characteristic rhythm. This strategy sends stimuli on each channel with its own characteristic rhythm. In order to do this, the same transform as in the first strategy is used, except that all the samples of the decomposition are kept from a scale to another. In that way, decomposition stages with frequency bands identical to those used in the first strategy are obtained but with the same number of samples for each stage as in the original signal. These coefficients are then sampled at a rate equal to the characteristic rhythm of the associated channel. This corresponds to an arbitrary sampling of the wavelet decomposition samples in this stage. The number of coefficients that will be kept depends, therefore, on the characteristic rhythm of the associated channel. The completeness of such sampling does not matter, contrary to the magnitude resolution of the wave, since, for some patients, the use of high stimulation rates can rapidly saturate the nerve and then restrict the dynamic range of the electric stimuli.

A fourth stimulation strategy stems from the fact that, sometimes, the wavelet decomposition coefficients in a given stage of the decomposition have very high magnitudes. In such cases, these coefficients cannot fit within the electric dynamic range of the associated channel. To solve this problem, part of the magnitude in a channel of high coefficient is transferred to a subsequent channel, thereby mimicking the accentuation effect performed by the external hair cells. This strategy uses the same stimulation rates as those used in the first strategy. The energy part in excess for a pulse in one channel is added to the energy of the pulse in the subsequent channel and so on.

Having described the system of the present invention in the form of preferred embodiments thereof, it will be appreciated that the cochlear prosthesis system and methods described herein rely on a new concept and benefit from a variety of innovative aspects.

The present device is highly versatile, and fully programmable. It can therefore address the needs related to a variety of pathologies, and can be easily upgraded, so that the patient is given the opportunity to benefit from any development in the field.

Incidentally, it can also be seen as a powerful tool for audiologists to discover new stimulation algorithms that would lead to a better comprehension of sound.

Moreover, the device of the present invention benefits from new sound signal processing techniques and new stimulation strategies that can be adopted by other systems, in order to better adapt the device to the patient pathology, facilitate the rehabilitation process, and lead to better speech comprehension without having recourse to lip-reading. This will allow increasing the number of implantees, including other patient categories such as prelingually, perilingually-deafened people and young children.

An appropriate hardware as well as a very user-friendly, since graphical, interface clinical software support all these new aspects. The latter also uses a modular design allowing to limit its options to a specific set up or to enlarge its possibilities to include developments and system upgrades.

It is to be understood that the hardware platform of the present invention cannot be completely functional without the complementary software part. In fact, such a complementarity ensures the modularity and then the versatility of the system.

While the programmable neurostimulator concept has been described herein as a cochlear prosthesis, it is to be understood that the present invention is not restricted to this type of neurostimulation.

Although the present invention has been described hereinabove by way of preferred embodiments thereof, it can be modified, without departing from the spirit and nature of the subject invention as defined in the appended claims.

What is claimed is:

1. A programmable neurostimulator comprising an external part linked to an internal part designed to be implanted in a patient's body, said internal part having a plurality of outputs, each one of said plurality of outputs being provided with an independent control unit and memory and with a current source, and being connectable to an independent electrode, so that each one of a plurality of independent electrodes is independently selectable and addressable.

2. The programmable neurostimulator according to claim 1, comprising at least sixteen electrodes each provided with a control unit and memory and with a current source.

3. The programmable neurostimulator according to claim 1, wherein any one of said plurality of outputs can be selected and activated in a variety of combinations including individually, simultaneously and using a specific synchronism, to address any one, any set or any subset of said plurality of independent electrodes independently.

4. The programmable neurostimulator according to claim 3, wherein said variety of combinations allows to set said plurality of outputs in a desired stimulation mode selected in the group comprising a monopolar stimulation mode, a bipolar stimulation mode, a quadripolar stimulation mode and an n-polar stimulation mode.

5. The programmable neurostimulator according to claim 1, wherein each one of said plurality of outputs is independently configurable as a current source, a current sink and a ground, and in a high impedance state, thereby allowing to generate a variety of current waveforms of a variety of shapes.

6. The programmable neurostimulator according to claim 1, wherein each one, each set or each subset of said plurality of outputs is independently configurable as a current source, a current sink, a ground, and in a high impedance state, thereby allowing to perform a monopolar mode, a bipolar mode, a quadripolar mode, and an n-polar stimulation mode, and to generate a variety of current distributions.

7. The programmable neurostimulator according to claim 6, wherein each one of said one, said set or said subset of the plurality of outputs can be activated individually, simultaneously and using a specific synchronism.

8. The programmable neurostimulator according to claim 1, wherein said internal part is based on an integrated circuit having a mixed-signal structure.

9. The programmable neurostimulator according to claim 8, wherein said integrated circuit includes a current level controller providing a current in each one of said plurality of outputs with a 8-bit accuracy, thus defining 32 current levels ranging from 0 to a maximum value selected by external logic signals.

10. The programmable neurostimulator according to claim 8, wherein said integrated circuit includes a processing logic unit controlling said plurality of outputs.

11. The programmable neurostimulator according to claim 8, wherein said integrated circuit comprises a digital part and an analogue part, said digital part controlling said analogue part by executing a set of command words.

12. The programmable neurostimulator according to claim 11, wherein said digital part includes means to decode said command words.

13. The programmable neurostimulator according to claim 11, wherein each one of said command words includes an address of at least one of said electrodes and a current level and polarity of said output sources.

14. The programmable neurostimulator according to claim 11, wherein said digital part includes a monopolar switch controller allowing to generate monopolar and bipolar stimulations individually and simultaneously.

15. The programmable neurostimulator according to claim 1, wherein said external part has a digital architecture supporting a plurality of signal processing methods and a plurality of stimulation strategies, thereby providing a plurality of stimulation methods by combining said plurality of signal processing methods with said plurality of stimulation strategies.

16. The programmable neurostimulator according to claim 15, wherein said external part is designed independently of said plurality of signal processing methods and of said plurality of stimulation strategies, so as to be adaptable to any digital architecture of said internal part.

17. The programmable neurostimulator according to claim 15, wherein said external part includes an external selection switch allowing selecting one of said plurality of stimulation methods.

18. The programmable neurostimulator according to claim 15, wherein said plurality of stimulation methods is based on an aspect of a speech signal included in the group comprising a frequency aspect of the speech signal, a temporal aspect of the speech signal and a mixed frequency and temporal aspect of the speech signal.

19. The programmable neurostimulator according to claim 15, wherein said plurality of stimulation methods includes single-rate, multi-rate, single-resolution and multi-resolution stimulation methods.

20. The programmable neurostimulator according to claim 19, wherein said multi-rate stimulation method allows a plurality of stimulation rates for a given stimulation strategy, and said multi-resolution stimulation method allows a plurality of different analysing resolution depending on details of the speech signal to be extracted.

21. The programmable neurostimulator according to claim 15, wherein said plurality of stimulation methods is based on a method selected in the group comprising a filter bank method, a vector quantization method and a wavelet packet method.

22. The programmable neurostimulator according to claim 21, wherein said stimulation method based on a filter bank method allows selecting an unlimited number of frequency sub-bands at a desired position and with a desired bandwidth, and associating a given stimulation channel to any one of said frequency sub-bands.

23. The programmable neurostimulator according to claim 21, wherein said stimulation method based on a vector quantization method comprises performing a fast spectral analysis of each speech segment, comparing obtained spectra to those of a codebook stored in the system memory, identifying the obtained spectra, and associating them to an element of the codebook, so as to generate a stimulation with an enhanced signal to noise ratio.

24. The programmable neurostimulator according to claim 21, wherein said stimulation method based on a wav let packet method allows a rate-place encoding of tonotopic information contained in the speech signal (frequency aspect), as well as a time-place encoding of fine temporal information (temporal aspect) thereof.

25. The programmable neurostimulator according to claim 24, wherein said stimulation method is a multi-resolution method that automatically adapts to characteristics of a detected sound, as well as to each patient's condition and pathology.

26. The programmable neurostimulator according to claim 24, wherein said stimulation method uses an unlimited number of mother wavelets.

27. The programmable neurostimulator according to claim 24, wherein one of said plurality of stimulation strategies consists in using different temporal resolutions for different stimulation sites and for different frequency ranges.

28. The programmable neurostimulator according to claim 24, wherein one of said plurality of stimulation strategies consists in using a maximal decomposition coefficient in each stage to modulate a pulse on a corresponding channel.

29. The programmable neurostimulator according to claim 24, wherein one of said plurality of stimulation strategies consists in sending stimuli on each channel with an own characteristic rhythm thereof.

30. The programmable neurostimulator according to claim 24, wherein one of said plurality of stimulation strategies consists in transferring part of the magnitude in a channel of high coefficient to a subsequent channel, so that an energy part in excess for a pulse in one channel is added to the energy of a pulse in a subsequent channel.

31. The programmable neurostimulator according to claim 1, wherein said external part operates in a mode selected in the group comprising a stand-alone mode and a slave mode.

32. The programmable neurostimulator according to claim 31, wherein said slave mode allows programming said external part by using a personal computer.

33. The programmable neurostimulator according to claim 1, wherein said neurostimulator is a cochlear prosthesis system.

34. The programmable neurostimulator according to claim 1, further comprising a clinical software psycho-acoustic test part, designed independently of a number and of an address of said plurality of outputs.

35. The programmable neurostimulator according to claim 1, wherein said external part makes use of a plurality of graphical windows, each one of said graphical windows being associated to a specific setup of a sound.

36. A programmable neurostimulator comprising an internal part and an external part, wherein said internal part comprises a digital portion and an analogue portion, said digital portion controlling said analogue portion by executing a set of command words, said internal part being built around an integrated circuit having a plurality of electrodes as current outputs channels; said external part supporting a plurality of sound processing methods and a plurality of stimulation strategies, so that said programmable neurostimulator allows a plurality of stimulation methods by combining said plurality of sound processing methods with said plurality of stimulation strategies.

37. The programmable neurostimulator according to claim 36, wherein each one of said plurality of electrodes is provided with an independent control unit, an independent memory unit and an independent current source so that any one of said plurality of electrodes can be associated with at least one other of said plurality of electrodes to form a stimulation channel.

38. The programmable neurostimulator according to claim 37, wherein said independent memory unit specifies an amplitude and a polarity of the current and specifies a state of one of said plurality of current outputs channels selected in the group comprising an active state, a ground state and a state of high impedance.

39. The programmable neurostimulator according to claim 37, wherein said independent control unit is a digital to analog (D/A) converter.

40. The programmable neurostimulator according to claim 36, wherein each one of said plurality of current output channels of the integrated circuit can be independently configured as a current source, a current sink, a ground or be set in a high impedance state.

41. The programmable neurostimulator according to claim 36, wherein each one of said plurality of current output channels of the integrated circuit can be selected and activated in a variety of combinations to independently address any of said plurality of electrodes, set of said plurality of electrodes and subset of said plurality of electrodes..

42. The programmable neurostimulator according to claim 36, wherein said plurality of stimulation methods comprises multi-rate and multi-resolution stimulation methods.

43. The programmable neurostimulator according to claim 36, wherein said neurostimulator is a cochlear prosthesis system.

44. A stimulation method for a neurostimulator, wherein said stimulation method includes a sound processing technique and a stimulation strategy.

45. The stimulation method according to claim 44, wherein said stimulation method makes use of a bank of filters associated with channels to generate a stimulation frequency for each one of said channels.

46. The stimulation method according to claim 44, wherein said stimulation method is based on a vector quantization technique whereby a finite set of elements of a sound is used for defining characteristics of a speech, in such a way that a patient is exposed to a limited number of stimulation sequences allowing a complete identification of said speech.

47. The stimulation method according to claim 44, wherein said stimulation method is based on a wavelet packet technique providing that a decomposition basis for a wavelet packet transform may be selected according to a given patient's perception of sound, on a pathological state of his cochlea, and on a current state of a surgical installation used.

48. The stimulation method according to claim 47, wherein said stimulation method uses a stimulation strategy whereby, progressing down from a scale to a following scale in a decomposition tree, a number of stages is doubled so that the frequency resolution gets higher and the number of samples, in each level of the decomposition, is kept the same as the number of original Input samples, In such a way that each one of the stimulation channels is associated with a stage in the decomposition tree; the strategy using different stimulation rates from one level to the other, and the rate of stimulation on each channel being fixed by a number of coefficients of the wavelet packet decomposition issued from the signal decomposition at an associated stage.

49. The stimulation method according to claim 48, wherein said coefficients of the wavelet packet decomposition at the associated stages and their temporal location dictate a time and an order of stimulation on each channel.

50. The stimulation method according to claim 47, wherein a maximal decomposition coefficient of said wavelet packet transform in each stage of the decomposition is used to modulate a pulse on a corresponding channel.

51. The stimulation method according to claim 47, wherein all the samples of the decomposition in the wavelet transform are kept from a scale to another, and the decomposition coefficients are sampled at a rate equal to a characteristic rhythm of an associated channel, so that the number of coefficients that is kept depends on the characteristic rhythm of the associated channel.

52. The stimulation method according to claim 47, wherein part of the magnitude in a channel of high coefficient is transferred to a subsequent channel, so that an energy part in excess for a pulse in one channel is added to the energy of a pulse in a subsequent channel.

53. A signal analyser having a modular architecture using, and independent of, a plurality of stimulation strategies as part of stimulation methods and built around a digital signal processor, comprising:
an operating system, which allows controlling general tasks;
a signal-processing unit to analyze a signal and to determine different aspects thereof to be taken into account;
a stimulation strategy unit to represent said aspects of the signal; and
an encoding unit of the system output;
wherein anyone of said functional parts can be programmed independently of the others.

54. The signal analyser according to claim 53, further provided with:
a graphical window dedicated to psycho-acoustic tests, which allows determining mapping parameters that are used by said stimulation methods;
a graphical window associated to each one of said stimulation methods, which permits to set up respective specific parameters of said stimulation methods;
wherein said graphical windows can communicate between each other for exchanging common specified data or interdependent set-ups.

55. The signal analyser according to claim 53, wherein said signal is a sound.

56. The signal analyser according to claim 54, wherein said signal analyzer provides a systematic phoneme identification process based on operations with a well-defined and limited number of frequency spectra, said frequency spectra including sound and noise so that said signal analyzer adapts to a variety of mother tongues and regional linguistic particularities.

57. The signal analyzer according to claim 53, wherein said signal analyzer is based on a auditory system modeling and on a representation of information in auditory nerves, and wherein said signal analyzer takes into account both frequency and temporal aspects of the signal.

58. The signal analyzer according to claim 53, wherein said signal analyzer is based on a auditory system modeling and on a representation of information in auditory nerves, and permits a rate-place encoding of tonotopic information contained in the signal as well as a time-place encoding of fine temporal information.

59. A cochlear prosthesis comprising an internal part to be implanted in an inner ear of a patient linked to an external sound analyzer, wherein said external sound analyzer is completely digital and uses a stimulation method comprising a sound processing technique sustaining a stimulation strategy to represent the sound, and an encoding unit of the sound so represented to be conveyed to the inner ear.

60. The cochlear prosthesis according to claim 59, wherein said sound processing technique is selected in the group comprising speech features extraction and wide-band speech processing.

61. The cochlear prosthesis according to claim 59, wherein said stimulation method is based on a technique selected in the group comprising a filter bank technique, a vector quantization technique and a wavelet packet technique.

62. The cochlear prosthesis according to claim 58, wherein said stimulation method makes use of a bank of filters associated with channels to generate a stimulation frequency.

63. The cochlear prosthesis according to claim 62, wherein said sound processing method allows to split a frequency band of the sound signal into as many different frequency sub-bands as is necessary and to vary a bandwidth and a central frequency of each frequency sub-band.

64. The cochlear prosthesis according to claim 59, wherein said stimulation method is based on a vector quantization technique whereby a finite set of elements of a sound is used for defining characteristics of a speech, in such a way that a patient is exposed to a limited number of stimulation sequences allowing a complete identification of said speech.

65. The cochlear prosthesis according to claim 64, wherein said stimulation method allows to perform a spectral analysis of each segment of the sound signal, to identify and associate each segment of the sound signal to an element of a pre-stored codebook of sound identification spectra so as to generate a corresponding stimulation sequence in the inner ear through commands sent to said internal part of the cochlear prosthesis.

66. The cochlear prosthesis according to claim 59, wherein said stimulation method is based on a wavelet packet technique providing that a decomposition basis for a wavelet packet transform may be selected according to a given patient's perception of sound, on a pathological state of his cochlea, and on a current state of a surgical installation used.

67. The cochlear prosthesis according to claim 66, wherein said stimulation method uses a stimulation strategy whereby, progressing down from a scale to a following scale in a decomposition tree, a number of stages is doubled so that the frequency resolution gets higher and the number of samples, in each level of the decomposition, is kept the same as the number of original input samples, in such a way that each one of the stimulation channels is associated with a stage in the decomposition tree; the strategy using different stimulation rates from one level to the other, and the rate of stimulation on each channel being fixed by a number of coefficients of the wavelet packet decomposition issued from the signal decomposition at an associated stage.

68. The cochlear prosthesis according to claim 67, wherein said coefficients of the wavelet packet decomposition at the associated stages and their temporal location dictate a time and an order of stimulation on each channel.

69. The cochlear prosthesis according to claim 66, wherein said stimulation method uses a maximal decomposition coefficient of said wavelet packet transform in each stage of the decomposition to modulate a pulse on a corresponding channel.

70. The cochlear prosthesis according to claim 66, wherein said stimulation method uses all the samples of the decomposition in the wavelet transform kept from a scale to another, and the decomposition coefficients are sampled at a rate equal to a characteristic rhythm of an associated channel, so that the number of coefficients that is kept depends on the characteristic rhythm of the associated channel.

71. The cochlear prosthesis according to claim 66, wherein said stimulation method transfers part of the magnitude in a channel of high coefficient to a subsequent channel, so that the energy part in excess for a pulse in one channel is added to the energy of the pulse in the subsequent channel and so on.
